(19) United States
(12) Patent Application Publication
(10) Pub. No.: US 2009/0247894 A1
(43) Pub. Date: Oct. 1, 2009

(54) Systems and Methods For Neurological Evaluation and Treatment Guidance

(75) Inventor: Elvir Causevic, New York, NY (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BrainScope Company, Inc.

(21) Appl. No.: 12/059,014

(22) Filed: Mar. 31, 2008

(51) Int. Cl. A61B 5/0484

(52) U.S. Cl. 600/544

(57) ABSTRACT

A system for acquiring and processing a subject's brain electrical activity is provided. The system includes at least one electrode, at least one analog amplifier channel, an analog-to-digital converter, a stimulus generator, and a digital signal processor configured to perform a harmonic signal analysis algorithm. The at least one analog amplifier channel, the analog-to-digital converter, and the digital signal processor are configured on a single integrated physical circuit. All the components of the system are configured to reside on a common portable unit to make the system easily applicable at the point-of-care. The system records and processes a subject's spontaneous neuroelectric signals as well as evoked potentials in real-time, and generates analyzed data representative of a subject's neurophysiological condition.

### FIELD OF THE INVENTION

The present invention generally relates to a medical apparatus, and more particularly, to a method and system for acquiring and processing brain electrical signals using an integrated, portable device.

### BACKGROUND OF THE INVENTION

Certain neurological disorders or conditions can be diagnosed by analyzing electrical signals from the brain using non-invasive tools, such as electroencephalography (EEG). A traditional brain wave recording system measures electrical potentials between electrodes placed on the scalp and generates a record of the electrical activity of the brain. Typically, such electrical activity may be shown as a set of analog waveforms or signals that must be interpreted by skilled neurophysiologists. This process can be time-consuming, expensive, technically demanding and subject to human error. Further, because results are not rapidly available, the traditional systems for analyzing brain electrical activity are not well suited for use in emergency rooms or other point-of-care settings.

Portable, easy-to-administer devices for recording and analyzing brain electrical activity would be beneficial in a number of clinical settings. For example, such devices would allow emergency response personnel to quickly evaluate patients with potential neurologic injury to allow rapid and proper initiation of therapy. For example, patients may present with a similar set of signs and symptoms when experiencing ischemic or hemorrhagic stoke. However, the proper therapies for ischemic and hemorrhagic disorders are vastly different, and improper or untimely differentiation between the two can be life-threatening. Therefore, a portable, rapidly-administered system for identifying these or many other neurologic conditions would be invaluable for rapid, on-site neurological evaluation.

Presently, portable brain wave recording systems may measure a subject's brain electrical impulses and convert them into digital data for transmission and downstream analysis. Such systems may additionally perform other steps in the external signal processing module, including further processing and analyzing the data, diagnosing the subject's condition, and displaying the resulting diagnosis. Results are typically displayed on a hand-held control distant from the patient. An exemplary system is disclosed in U.S. Pat. No. 6,052,619 and related U.S. application Ser. No. 10/045,799, both of which are incorporated herein by reference. Such a system generally features a headband with an array of electrodes configured to detect, amplify, and broadcast data, via radio or cellular phone, to a local receiver for analysis. Such a system may also record evoked potentials following administration of a stimulus. The system processes data using various tools, including traditional Fast Fourier Transform (FFT) analysis and power spectral density (PSD) analysis.

Alternate signal processing tools, such as Harmonic Signal Analysis, have been used advantageously in the analysis of brain electrical activity, and have been successfully applied to neurological evaluation. Such tools and systems are disclosed in U.S. Patent Publication No. 2007/0032737 A1 (application Ser. No. 11/195,001), incorporated herein by reference.

The inventor of the present invention has recognized the need for a portable, easy-to-use, low-cost, low-power system for acquiring and processing brain electrical signals and displaying a diagnosis in real-time. Inefficiencies in existing integrated circuit technologies are prohibitive to creating a single, integrated chip for acquiring and processing analog neuroelectric signals. This is because brain signal acquisition and processing requires very high precision, more power, and more physical space, and therefore, integrated circuits for processing brain electrical signals have not been developed. The current invention presents a novel system for neurological evaluation that integrates signal amplification, analog-to-digital conversion, and digital signal processing on a single, standalone chip, with all the components fabricated on the same die, and running on the same clock, at the same temperature, same parasitic capacitance, and same ground plane, which helps to reduce noise, power dissipation and allows high speed.

### SUMMARY OF THE INVENTION

The present disclosure provides methods and systems for acquiring, processing, and analyzing brain electrical activity for evaluating the neurophysiological condition of the brain. Methods and systems for improving the acquisition and processing of analog bioelectric signals are disclosed. Advantages of the present invention may include, but are not limited to, reducing the size of the system, improving portability, facilitating integration, enabling high-speed, real-time processing, reducing noise contamination, enabling the acquisition and processing of submicrovolt signals, reducing production costs, and reducing complexity of system deployment.

One embodiment consistent with the principles of the invention is a system for acquiring and processing a subject's brain electrical activity using Bx™ technology. The neurological evaluation system includes at least one electrode, at least one analog amplifier channel, an analog-to-digital converter (ADC), a stimulus generator for eliciting evoked potentials, and a digital signal processor (DSP) to implement harmonic signal analysis-based signal processing. The at least one analog amplifier channel, ADC converter, and the digital signal processor are configured to reside in a single integrated physical circuit. For multi-channel applications, the system also comprises an analog multiplexer, which is included in the single integrated circuit

Other embodiments consistent with the principles of the invention include a method for determining the neurological state of a subject and a system for the same. The method includes the steps of providing an integrated device for measuring and processing brain electrical activity, attaching an electrode array to a patient, activating a stimulus generator, and detecting data relating to the subject's spontaneous brain electrical activity and evoked potentials generated in response to applied stimuli. The integrated device includes an analog module and a DSP module that performs, for example a harmonic signal analysis algorithm, to process the data representative of the acquired brain electrical impulses.

Additional embodiments consistent with the principles of the invention include methods for analyzing data relating to brain electrical signals and evoked potentials, and a system for the same. The method includes the steps of bit-level processing, artifact detecting, feature extracting, classifying, and displaying output.

Additional embodiments consistent with principles of the invention are set forth in the detailed description which follows or may be learned by practice of methods or use of systems or articles of manufacture disclosed herein. It is understood that both the foregoing general description and the following detailed description are exemplary and explanatory only, and are not restrictive of the invention as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. In the drawings:

FIG. 1A illustrates an exemplary embodiment of a neurological evaluation system, consistent with features and principles of the present invention.

FIG. 1B illustrates an exemplary embodiment of the neurological evaluation system of FIG. 1A interfacing with a patient, consistent with features and principles of the present invention.

FIG. 2A illustrates a block diagram of an exemplary integrated chip for brain signal acquisition and processing, consistent with features and principles of the present invention.

FIG. 2B illustrates a block diagram of the neurological evaluation system of FIG. 1A-1B, consistent with features and principles of the present invention.

FIG. 3 illustrates a block diagram of an exemplary analog module, as may be included in an embodiment of the neurological evaluation system of the present disclosure, consistent with features and principles of the present invention.

FIG. 4 illustrates a block diagram of an exemplary digital signal processor, as may be included in an embodiment of the neurological evaluation system of the present disclosure, consistent with features and principles of the present invention.

FIG. 5 illustrates an exemplary analog integrated circuit, as may be included in an embodiment of the neurological evaluation system, consistent with features and principles of the present invention.

FIG. 6 illustrates an exemplary digital signal processing integrated circuit, as may be included in an embodiment of the neurological evaluation system, consistent with features and principles of the present invention.

FIG. 7 illustrates an exemplary method for determining a patient's neurological state, consistent with the features and principles of the present invention.

### DESCRIPTION OF ILLUSTRATIVE EMBODIMENTS

Reference is now made in detail to exemplary embodiments of the invention, examples of which are illustrated in the accompanying drawings. Wherever possible, the same reference numbers will be used throughout the drawings to refer to the same or like parts.

In the following detailed description, reference is made to the accompanying drawings, which form a part hereof and in which is shown by way of illustration specific embodiments in which the invention may be practiced. These embodiments are described in sufficient detail to enable one skilled in the art to practice and use the invention, and it is to be understood that other embodiments may be utilized and that electrical, logical, and structural changes may be made without departing form the spirit and scope of the present invention.

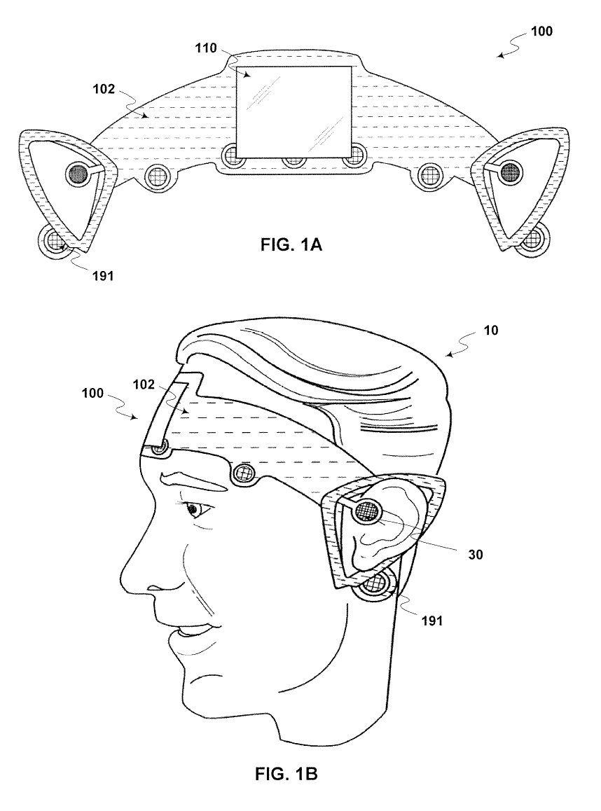

FIG. 1A illustrates an exemplary embodiment of a neurological evaluation system 100 using Bx™ technology. The components of system 100 may be positioned on a headband/headgear 102 that can be attached to a patient 10. System 100 and its components are described in detail below.

FIG. 1B illustrates an exemplary embodiment of system 100, as it may be practiced. As shown, system 100 can be attached to a patient 10 using headband 102, which allows one or more electrodes 191 to be attached to the forehead of patient 10. In some embodiments, system 100 can be attached to patient 10 by looping headband 102 around the ears of patient 10, as shown. However, it will be appreciated that alternative configurations are possible and are within the scope and principles of the present disclosure. For example, any suitable support and/or attachment structure may be used to facilitate quick and secure attachment of system 100 to a patient to facilitate acquisition of data pertaining to brain electrical activity. The neurological evaluation system 100 can be sold and distributed as a single, ready-to-use, or nearly ready-to-use system, and can be fabricated at relatively low cost to allow disposability after one or several uses.

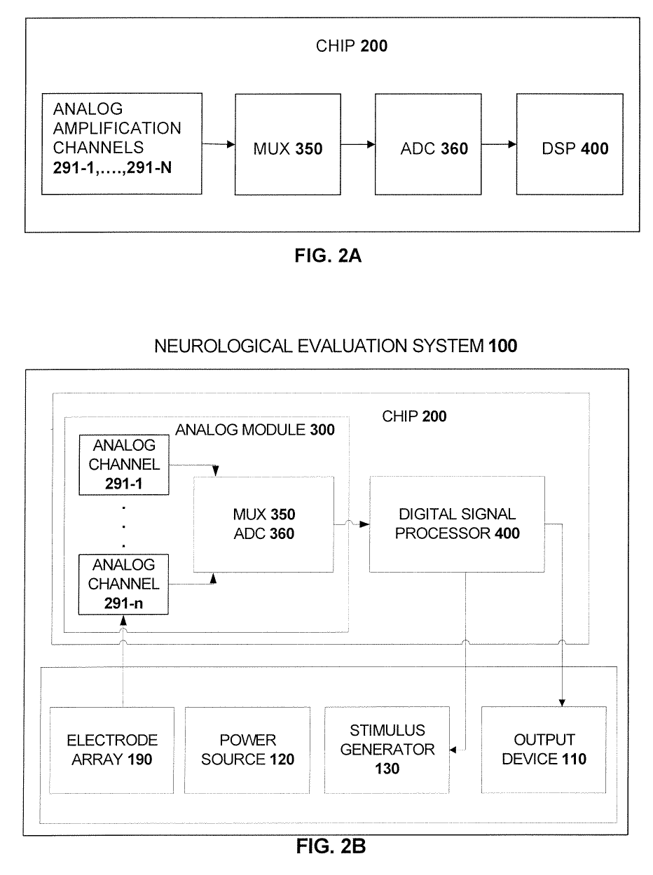

Further, as described in detail below, system 100 can include a number of components. For example, in one embodiment, system 100 can include a single-chip circuit system 200, henceforth referred to as chip 200, as described with respect to FIGS. 2A and 2B. FIG. 2A illustrates an exemplary embodiment of chip 200, and FIG. 2B shows chip 200 interfacing with other electrical components pertinent to brain signal acquisition, processing and display of results. Chip 200 can include a number of important electrical components, including for example, at least one analog amplifier channel, an analog multiplexer for multi-channel applications, an analog-to-digital converter (ADC), and a digital signal processor, as described below. In a embodiment consistent with the present invention, chip 200 may also include a digital-to-analog converter (DAC) to convert the processed data into analog waveforms that represent brain electrical activities, or to send digital input data to the signal acquisition components The inclusion of certain electrical components in chip 200 can provide a number of advantages. For example, chip 200 can be quickly and easily attached to other components, such as an electrode array and/or supporting headband, along with electrical components contained therein. The chip 200, the electrode array 190 and/or supporting headband may also be sold together as a kit for point-of-care applications. Additional advantages of the present invention may further include improvements in portability, ease-of-use, feasibility of mass production, and ability to acquire and process submicrovolt signals in real time. The electrical components of chip 200 can be operatively coupled with other components, such as a stimulus generator 130 and output device 110.

Neurological evaluation system 100 can be a standalone system or can operate in conjunction with a mobile or stationary device to facilitate display or storage of data, and to signal healthcare personnel when therapeutic action is needed, thereby facilitating early recognition of alarm conditions. For example, system 100, operating in conjunction with a mobile or stationary telemetry or monitoring system, as may be available in hospitals, can cause the mobile or stationary system to trigger an alarm and/or notify medical personnel to respond to some neurological conditions. Mobile devices can include, but are not limited to, handheld devices and wireless devices distant from, and in communication with, system 100. Further, stationary devices can include, but are not limited to, desktop computers, printers and other peripherals that display or store the results of the neurological evaluation. The system may communicate wirelessly with the mobile or stationary devices, and in which case, the system 100 may also include a wireless output interface.

Further, system 100 can transmit data to another mobile or stationary device to facilitate more complex data processing or analysis. For example, system 100, operating in conjunction with a desktop computer, can send data to be further processed by the computer. Additionally, system 100 can be configured to interact with a printer or other system to print or store medical records, and therefore, may be configured to automatically generate medical records to be stored or used by attending medical personnel.

Referring again to FIG. 2, analog module 300 of the chip 200 may receive signals from one or more system electrodes 191-1, . . . , 191-n, operatively connected through analog channels 291-0, . . . , 291-n+1. Further, analog module 300 may be configured to amplify, filter, and preprocess analog waveforms acquired from each channel 291, as described in detail below. The analog module 300 may further include a multiplexer (MUX) 350, which combines many analog input signals and outputs that into a single channel, and an analog-to-digital converter (ADC) 360 to digitized the received analog signal. Digital signal processor 400 can process digitized data acquired through analog module 300 and can perform analysis of data to aid in interpretation of data pertaining to brain electrical activity. Further, as noted above, the components of chip 200 may be operatively connected with a number of additional device components. For example, as shown and described in detail below, neurological evaluation system 100 may include an output device 110.

In an exemplary embodiment of system 100, output device 110 may be configured to communicate information or test results about patient 10 to other devices or personnel, such as an attending physician, an emergency response or medical technician, a computer, or a server. Information that is conveyed through output device 110 can include a variety of different data types, including, but not limited to, raw data, encoded data, signal waveforms, diagnostic results, intermediate analysis results, alarms, alarm conditions, usage settings, etc. In some exemplary embodiments, output device 110 may receive and display usage setting information, such as the name, age and/or other statistics pertaining to patient 10. Additionally or alternatively, output device 110 may be configured to display brain electrical activity waveforms of patient 10. Subsequently, output device 110 may display an indicator representing the condition of patient 10. This and other embodiments are within the scope of the present invention.

Output device 110 may present results in various forms, including, for example, auditory and visual forms. Visual results may be presented through any suitable visual display, such as a liquid crystal display (LCD) or a touch screen, but it will be understood by one skilled in the art that many other presentation devices exist and may be used in conjunction with embodiments of the present invention. For example, output device 110 may present results through automated speech.

Output device 110 may also be contained within the portion of system 100 that is attached to the forehead of patient 10, thereby allowing the device to be contained in a single unit and allowing medical personnel treating the patient 10 to see the results as they examine the patient or attend to other patient needs. The output device may be a visual display or an LED that lights up when immediate medical attention is required. Alternatively or additionally, output device 110 may be contained in a separate system or housing such as a remote monitor near a nurse's station, and may interface with system 100 wirelessly. In an exemplary embodiment, chip 200 may also contain a wireless power amplifier coupled directly to an antenna to transmit diagnostic results wirelessly to the output device or to a remote data storage system.

As shown, output device 110 can interface with digital signal processor 400. In such embodiments, output device 110 may present diagnostic results produced by processor 400. In other embodiments, output device 110 may alternatively or additionally interface with analog module 300. In such embodiments, output device 110 may display raw or quantized data prior to analysis.

As noted, neurological evaluation system 100 can include a power source 120 to enable the operation of all the components of system 100. Power source 120 can include any voltage or current source, including, but not limited to, a variety of batteries or alternating current sources. In some exemplary embodiments, power source 120 can include a relatively low-power and/or short-life battery selected to minimize size, weight, and/or cost. In some embodiments, power may be transferred to the system wirelessly using electromagnetic coupling with an external source, in which case, the power source 120 may include an antenna to receive RF emissions from the external source and a capacitor circuit to store the received power. The power requirements of system 100 is considerably reduced by including several important components within a single chip 200 and by configuring system 100 to perform custom signal processing.

As shown, power source 120 can reside external to chip 200, and an interface may be provided between power source 120 and electrical components within chip 200. In alternate embodiments, power source 120 may be included in chip 200 and may be directly connected to components within chip 200.

While analysis generally may include the step of acquiring a subject's base brain electrical activity over a period of time, analysis may further include the extraction of evoked potential (EP) signals. EP signals are transient signals that contribute to a subject's overall brain electrical activity. EP signals are generally produced in response to the detection of external stimuli by the brain. In some embodiments, suitable systems can include one or more stimulus generators 130 to produce audio, visual or electrical stimuli that can elicit evoked potentials (EP) to be evaluated by system 100. The administration of stimuli can facilitate production of EP signals and aid in the diagnosis of certain neurological disorders. External stimuli can include auditory, visual, and electrical. Typically, the amplitude of EP signals are approximately one order of magnitude smaller than the base electrical signals.

The stimulus generator may interface with a stimulus output device, such as an audible stimulus output device 30, positioned proximate a patient's ear and configured to produce sounds that can generate EPs. In some embodiments, consistent with features and principles of the present invention, the stimulus generator 130 may also reside within the chip 200.

One subset of EP signals are auditory evoked potential (AEP) signals. AEP signals are elicited by administering auditory stimuli. AEP signals further comprise an auditory brainstem response (ABR), a mid-latency cortical response (MLR), and a slow cortical response. ABR generally occurs during the first 11 ms after the stimulus is administered.

In an exemplary embodiment, neurological evaluation system 100 may utilize the advantages of ABR signals to map specific auditory, neurological and psychiatric dysfunctions. In such an embodiment, stimulus generator 130 can administer auditory stimuli. As noted above, auditory stimuli can be administered by placing an auditory output device 30 in proximity to patient 10 such that the stimuli may be detected by the patient. Auditory stimuli can comprise discrete or continuous sound signals, or a combination of both. Examples of discrete sounds, which can be used with neurological evaluation system 100, include clicks and pulses. In some embodiments, the sounds can be administered as a succession of sounds at varying frequencies.

In an alternate exemplary embodiment, stimulus generator 130 may administer visual stimuli. Visual stimuli may be applied by placing a visual output device in proximity to patient 10, such that the stimuli may be detected by patient 10. Visual stimuli may comprise discrete or continuous visual signals, or a combination of both. Examples of discrete visual signals may include flashes of light and light of varying wavelengths.

As noted above, system 100 can further include electrode array 190, which may be configured to receive signals pertaining to neurological electrical activity so that such signals can be acquired and processed by system 100. Electrode array 190 can comprise any number of electrodes 191-1 . . . , 191-n arranged to facilitate acquisition of data pertaining to brain electrical activity. Many such arrangements exist, wherein the number of electrodes may range from about one to about twenty or more electrodes. Electrodes 191 can be positioned in a variety of locations on or near the head, including, but not limited to, the forehead, the scalp, the temples near the ears, and on the neck or upper back.

An example of such an arrangement known in the art is typically referred to as the standard 10/20 system. In the standard 10/20 system, raw data is collected from nineteen regions of the head using twenty electrodes positioned along the forehead and scalp of patient 10.

Another example of such an arrangement includes a configuration of nine electrodes covering the areas around the right mastoid, far right of the forehead, near right of the forehead, center top of the forehead, near left of the forehead, far left of the forehead, left mastoid, and left shoulder of patient 10. Such an arrangement is disclosed in U.S. Pub. No. 2007/0032737 A1, incorporated herein by reference in its entirety.

In some exemplary embodiments of electrode array 190, electrodes 191-1, . . . , 191-n are positioned primarily around the forehead. For example, electrode array 190 may include nine electrodes 191-1, . . . , 191-n positioned across the forehead and/or near the ears. It will be understood by one skilled in the art that alternative embodiments are within the scope of the disclosure and are consistent with features and principles of the present invention.

In some exemplary embodiments, electrode array 190, including electrodes 191-1, . . . , 191-n, may be contained within an attachable subset of system 100. For example, each of electrodes 191 can be individually or collectively connected to chip 200 to interface with components contained therein. Further, electrode array 190 may be configured to include hooks or nontoxic adhesive or conducting gel to enable noninvasive and reliable attachment to patient 10.

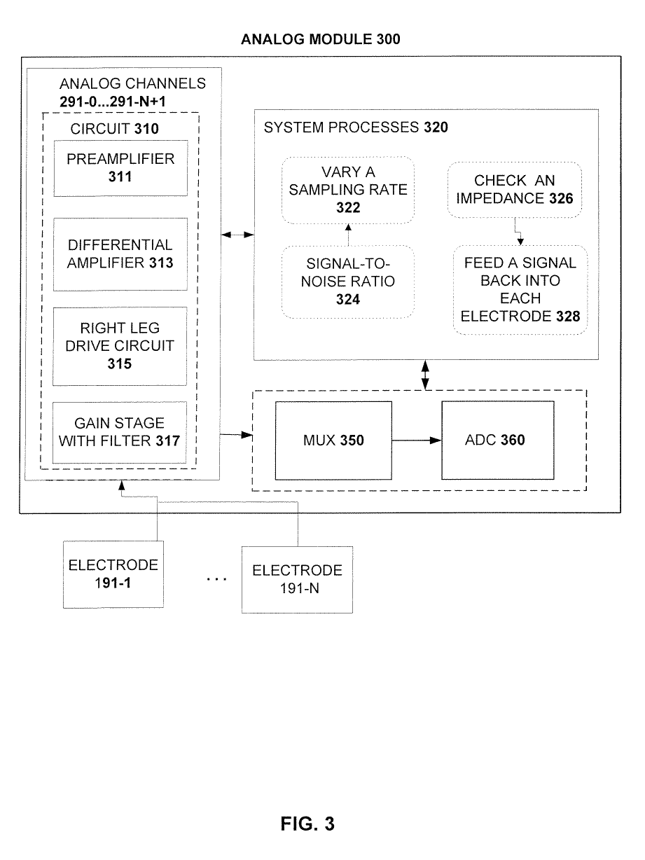

FIG. 3 illustrates a block diagram of components of exemplary analog module 300, as may be included in an embodiment of neurological evaluation system 100, consistent with features and principles of the present invention. As shown, analog module 300 comprises at least one analog amplifier channel 291-0, . . . , 291-n+1, which in turn interfaces with at least one electrode 191-1, . . . , 191-n. Analog amplifier channels include a circuit 310 including at least one preamplifier 311, at least one differential amplifier 313, at least one common mode detector 315, and at least one gain stage with filter 317. Analog amplifier channels 291-1, . . . , 291-n may correspond with data acquired from electrodes 191-1, . . . , 191-n. Further, analog channels 291-0 and 291-n+1 may be included as unused or unprocessed analog channels. Advantages of having unused analog channels 291-0 and 291-n+1 may include the ability to mitigate edge effects and asymmetry of signals acquired on analog channels 291-1, . . . , 291-n.

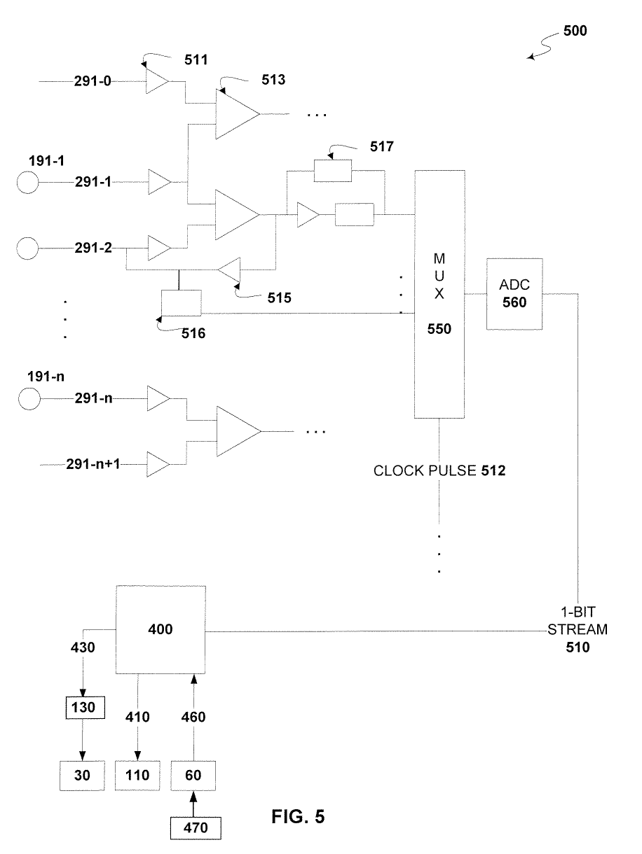

In some exemplary embodiments, analog module 300 can interface with digital signal processor 400 through an interface, such as a multiplexer (MUX). Further, a suitable interface may also comprise an analog-to-digital converter (ADC), such as a 1-bit sigma-delta analog-to-digital converter, to digitize the continuous analog signals for processing. Such configurations are described in detail with reference to FIG. 5. Referring again to FIG. 3, analog module 300 can be configured to perform system processes 320. In some exemplary embodiments, system processes 320 can include varying a sampling rate 322 based on a signal-to-noise ratio 324. Varying a sampling rate 322 can enable maximization of the sampling rate (oversampling), thereby reducing aliasing during sampling. Further, the reduction of signal aliasing may correspondingly relax the requirement on anti-aliasing filter transition band sharpness (hence, the filter complexity) at the expense of using a faster ADC, or may help to obviate the need for an anti-aliasing filter altogether.

In some exemplary embodiments, system processes 320 may include checking an impedance 326 by feeding a signal back into each electrode 328. Checking an impedance 326 may function to characterize the effectiveness of connection of a surface electrode to a subject. This would further enable the ability to test applied electrodes at patient site before connection to the system 100, and measure the electrode impedances continuously in real-time while a patient is being monitored. In some embodiments, the electrodes 191-0 . . . 191-n may further contain LEDs, which turn on when its impedance is higher than the preselected value. In another embodiment, the impedance of the applied electrodes may be displayed on the output device 110 connected to the digital signal processor 400.

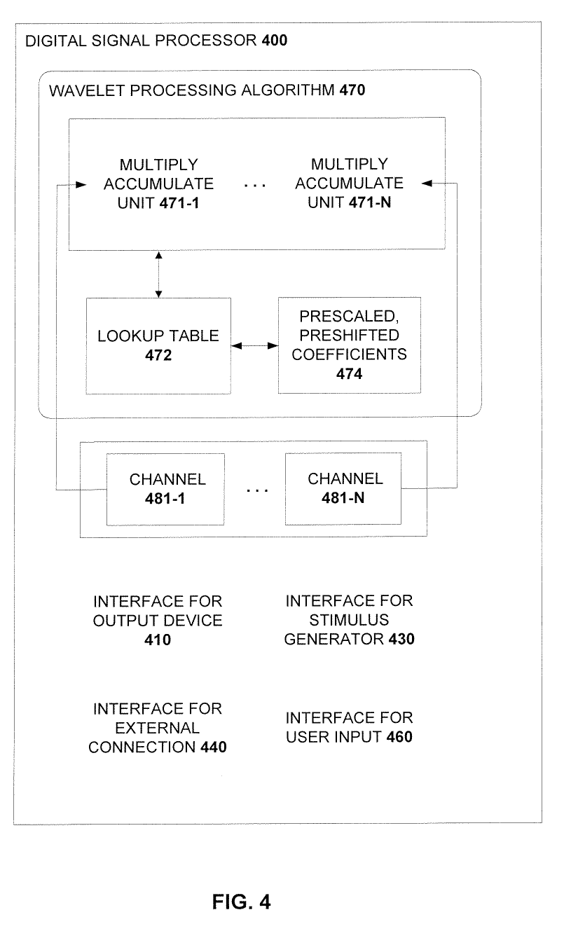

FIG. 4 illustrates a block diagram of exemplary digital signal processor 400, as may be included in an embodiment of system 100, consistent with features and principles of the present invention. It will be understood by those skilled in the art that there exist many digital signal processors that may be selected for use in the system 100, consistent with features and principles of the present invention.

In an exemplary embodiment, digital signal processor 400 may be configured to perform a harmonic signal analysis algorithm 470. Harmonic signal analysis algorithm 470 may comprise at least one of wavelet transform methods, such as wavelet packets, discrete wavelet transform, or diffusion wavelet transform. Wavelet processing methods are disclosed in U.S. Pat. No. 7,054,454, which is assigned to Everest Biomedical Instruments Company and is incorporated in its entirety herein by reference. The digital signal processor 400 may also be configured to perform fractal analysis and multiscale harmonic analysis.

In one exemplary embodiment, the digital signal processor 400 may include a plurality of multiply accumulate units (MAUs) 471-1, . . . , 471-N, lookup table 472, and prescaled, preshifted coefficients 474, as may be stored in lookup table 472. Advantages of custom signal processing integration, as such, include reduction of power consumption by digital signal processor 400.

In some exemplary embodiments, digital signal processor 400 may include N multiply accumulate units 471-1, . . . , 471-N, corresponding to N channels, 481-1, . . . , 481-N, that carry 1-bit data stream in parallel to the N MAUs. Advantages of parallel processing and distribution may include improved online, real-time data processing. The N channels, carrying the 1-bit data stream from the sigma-delta ADC converter, correspond to the analog neuroelectric signal received from the analog channels.

Referring again to FIG. 4, digital signal processor 400 can optionally include interfaces for various modules, including, but not limited to, an output device interface 410, a stimulus generator interface 430, an external connection interface 440, and a user input interface 460.

In some embodiments, output device interface 410 can include a bus that connects digital signal processor 400 with output device 110. The data that is sent along the bus, or other interface, such as a wireless connection, may be particularly suited for the type of output device 110 deployed. In some embodiments, stimulus generator interface 430 may connect a stimulus generator 130 to the digital signal processor 400, which controls the stimulus generator. In some embodiments, external connection interface 440 may be a wireless connection to a printer or a handheld or otherwise mobile device. Additionally, external connection interface 440 may comprise a direct wired connection to any external device.

User input interface 460 can connect system 100 to a user input device, such as a keyboard, through digital signal processor 400. In some embodiments, user input interface 460 can be incorporated in output device interface 410. For example, output device 110 can include a touch screen that is designed to both display end-user information and accept user input. In some embodiments, user input interface 460 may be incorporated with an interface for external connection 440. For example, interface for external connection 440 can enable a connection between system 100 and a mobile device or stationary computer 470, and the input interface 460 may be further configured to accept and transmit input data to digital signal processor 400. The external connection 440 may again be a direct wired or wireless connection.

FIG. 5 illustrates an exemplary integrated circuit 500, as may be included in neurological evaluation system 100, consistent with features and principles of the present invention. As shown, exemplary integrated circuit 500 can comprise electrodes 191-1, . . . , 191-n and analog channels 291-0, . . . , 291-n+1. Each analog channel includes a preamplifier 511, a differential amplifier 513 (one differential amplifier for a pair of electrodes), common mode detector 515 (right leg drive circuit), impedance measurement stage 516, and gain stage with filter 517.

As shown in exemplary integrated circuit 500, electrodes 191-1, . . . , 191-n acquire data relating to the brain electrical activity of patient 10 and feed the data into analog channels 291-1, . . . , 291-n. Additionally, exemplary integrated circuit 500 includes unused analog channels 291-0, 291-n+1, which serve as dummy or compensatory structures at the edge of the array of channels. Advantages of having the unused channels may include mitigation of errors caused by edge effects in the integrated circuit, and more particularly, to the mitigation of mismatches and non-uniformities between the electrical structures.

In some exemplary embodiments, integrated circuit 500 can comprise nine electrodes 191-1, . . . , 191-9 and eleven analog channels 291-0, . . . , 291-10, wherein data from analog channels 291-0, 291-10 are unused. Advantages of having nine electrodes 191-1, . . . , 191-9, as opposed to a standard 20/10 electrode set, can include reduced placement complexity.

In some embodiments of integrated circuit 500, the recording electrodes are not directly connected to the differential amplifier, but are connected by way of preamplifier stages 511, that may serve to amplify submicrovolt signals for processing in some embodiments. Such amplifiers may have high input impedances and low output impedances to increase the effective CMRR (Common Mode Rejection Ratio). Subsequent to preamplification, the signal may be passed through differential amplifier stages 513. Each differential amplifier has two inputs and an electrode is connected to each input via the preamplifier, in a manner called ‘montage’ which is known in the prior art. The differential amplifier measures the voltage difference between the two signals at each of its inputs, and the resultant signal is amplified.

In some embodiments, the amplified analog signal produced by differential amplifiers 513 can be used to check lead impedance 326 and a signal can be fed back to the electrode via the digital signal processor 400, as described by system processes 320 with reference to FIG. 3. It will be understood by one skilled in the art that signal feedback as described may generally be performed for each of the plurality of electrodes 191-1, . . . , 191-n.

In some embodiments, the analog signal produced by differential amplifiers 513 can be passed through a common mode detector 515 (a right leg drive circuit), which raises the CMRR of the system. This may reduce noise that appears as common-mode voltage signal on both input leads of the differential amplifier, thereby improving the signal-to-noise ratio of the signal processing component of system 100.

In some embodiments, integrated circuit 500 can additionally include a gain stage with filtering 517. The one or more filters used in stage 517 can include low pass, band pass, or high pass filters, depending on the algorithm employed by integrated circuit 500. In an exemplary embodiment of integrated circuit 500, as may be used in conjunction with neurological evaluation system 100.

Additionally, integrated circuit 500 can further include multiplexer (MUX) 550, an ADC 560 which can be 1-bit sigma-delta (ΣΔ) modulator without noise shaping, and a digital signal processor 400. MUX 550 may additionally be configured to output a channel timing 512 signal, which serves as the clock signal for the shift registers of digital signal processor 400. ADC 560 may transmit 1-bit stream 510 to processor 400. ADC 560 may also have a variable sampling rate, which enables oversampling to avoid aliasing.

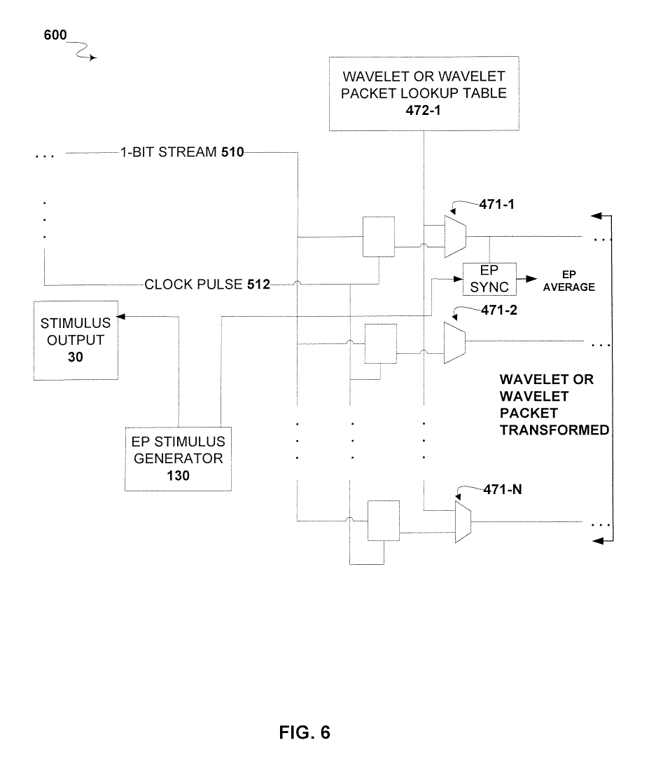

FIG. 6 illustrates an exemplary embodiment of digital processor 400, as may be included in the integrated circuit 500 of neurological evaluation system 100. As shown, exemplary processor 400 receives 1-bit stream 510 from the ADC 560 and clock pulse 512 from MUX 550.

Additionally, as shown in an exemplary embodiment, digital signal processor 400 can comprise a plurality of multiply accumulate units 471-1, . . . , 471-N with transform coefficient lookup table 610 per channel, a plurality of shift registers that help in handling the data processing, channels 481-1, . . . , 481-N that carry the 1-bit data stream from the ADC 560, and an EP sync block for synchronizing the timing of the operation of the processor 400 with the timing of generating stimulus pulses by the stimulus generator 130, since the EPs are time-locked to stimuli onset to improve signal-to-noise ratio. One of the input to the shift registers is the clock pulse input from the MUX 550, and data is shifted right (down) into the MAUs according to the clocking rate. The look-up tables store pre-scaled, pre-shifted coefficients, and the size of the look-up table is kept limited to the requirements of the wavelet algorithm to limit memory storage demands.

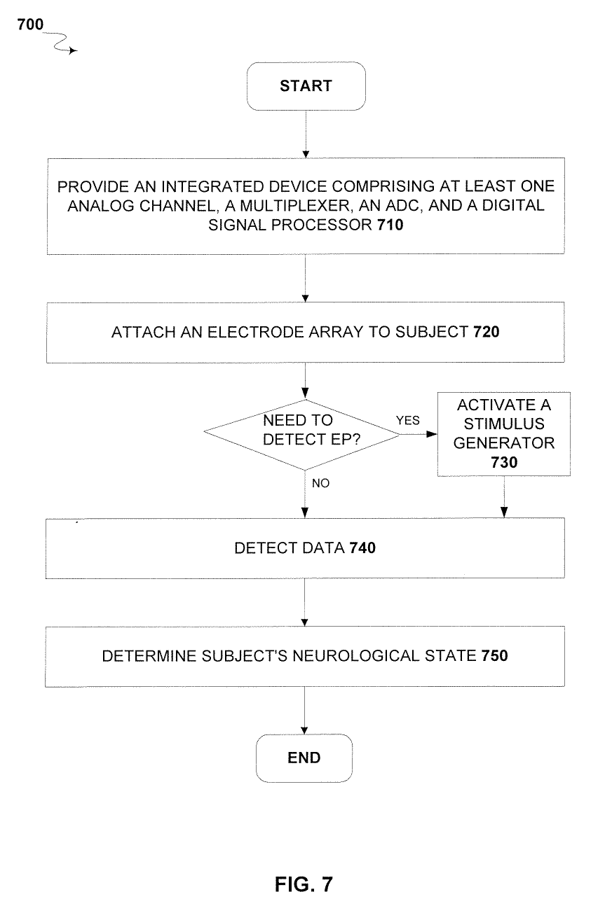

FIG. 7 illustrates an exemplary method for determining a subject's neurological state using Bx™ technology, consistent with the features and principles of the present invention. According to the method of the present disclosure, an integrated circuit system including at least one analog amplifier channel, an analog multiplexer for multi channel applications, an ADC, and a digital signal processor is provided, as indicated at Step 710. As previously disclosed with reference to FIG. 2, the neurological evaluation system may include additional components, including, but not limited to a stimulus generator, an output device and a power source.

Once selected, an electrode array may be attached to a subject, as indicated at Step 720. As disclosed with reference to FIG. 1, the electrode array may comprise at least one electrode and may be attached to the subject in a variety of ways.

Next, a stimulus generator may be activated if evoked potentials have to be recorded, as indicated at Step 730. The stimulus generator may be activated by the digital signal processor 400. The device provided at step 710 may require configuration prior to activating the stimulus generator at Step 730. Configuration requirements may include, but are not limited to, activating the device, providing user input, and establishing a connection to one or more external devices.

The activation of the stimulus generator at Step 730 may cause the subject's neurological activity to include EP activity. The device may detect data at Step 740. The data may comprise spontaneous brain electrical activity and evoked potentials.

Next, as shown in exemplary method 700, the device may continue to activate the stimulus generator and detect data in parallel, or the device may record the base brain electrical activity without eliciting evoked potential. The data is processed by the digital signal processor using harmonic signal analysis. The processor may also be configured to perform transform-domain denoising algorithm, which would efficiently remove both Gaussian as well as Gaussian mixed with impulse noise contamination. The processor may also be configured to perform fractal analysis and multiscale harmonic analysis to process the data. At Step 750, when the device has sufficient data to perform analysis, a determination may be made as to the subject's neurological state, which may be based on the analysis results. The method may continue to assess the subject's neurological state as additional data is acquired and processed over time.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for acquiring and processing brain electrical activity comprising:
at least one analog amplifier channel;
an analog-to-digital converter;
a digital signal processor configured to perform a signal processing algorithm to analyze the signal;
wherein the at least one analog amplifier channel, the analog-to-digital converter, and the digital signal processor reside in a single integrated physical circuit.

2. The system of claim 1, wherein said single integrated circuit is configured to produce a result.

3. The system of claim 1, further comprising an output device.

4. The system of claim 3, wherein said output device is operatively coupled to said single integrated circuit.

5. The system of claim 4, wherein said output device is directly connected to said single integrated circuit to display a result of one or more operations performed by said digital signal processor.

6. The system of claim 4, wherein said output device communicates wirelessly with said digital signal processor.

7. The system of claim 6, wherein said single integrated circuit further comprises a wireless power amplifier coupled to an antenna to transmit a result wireless to said output device

8. The system of claim 1, further comprising an electrode array including at least one electrode.

9. The system of claim 1, further comprising an analog multiplexer for applications using multiple said analog amplifier channels;
wherein said multiple analog amplifier channels, said analog multiplexer, said analog-to-digital converter, and said digital signal processor reside in a single integrated physical circuit.

10. The system of claim 1, further comprising a stimulus generator.

11. The system of claim 1, further comprising a power source.

12. The system of claim 11, wherein said power source receives power wirelessly from an external source.

13. The system of claim 8, wherein said electrode array including at least one electrode, and said single integrated physical circuit are configured to reside in a portable unit; and
wherein said portable unit further comprises a stimulus generator, a power source, and an output device.

14. The system of claim 13, wherein said portable unit can be in the form of a headgear to be mounted on a subject for acquiring and processing brain electrical activity.

15. The system of claim 1, wherein the system is configured to acquire and process data pertaining to spontaneous brain electrical activity.

16. The system of claim 1, wherein the system is configured to acquire and process data pertaining to spontaneous and evoked potentials.

17. The system of claim 16, wherein said evoked potentials are generated in response to stimuli from a stimulus generator.

18. The system of claim 1, wherein the at least one analog channel comprises a plurality of active analog channels.

19. The system of claim 1, wherein the at least one analog channel further comprises at least one analog channel not connected to an electrode to mitigate common temperature and silicon process effects.

20. The system of claim 1, wherein the at least one analog channel comprises at least one preamplifier, at least one differential amplifier, at least one gain stage with at least one filter, and at least one common mode detector.

21. The system of claim 1, wherein said analog-to-digital converter is a 1-bit sigma-delta modulator.

22. The system of claim 1, wherein said digital signal processor further comprises at least one multiply accumulate unit.

23. The system of claim 1, wherein said digital signal processor is configured to perform harmonic signal analysis.

24. The system of claim 1, wherein said digital signal processor further comprises at least one lookup table to store prescaled and preshifted signal transform coefficients.

25. The system of claim 23, wherein said digital signal processor is further configured to perform fractal analysis and multiscale harmonic analysis.

26. The system of claim 1, wherein said digital signal processor further comprises of an evoked potential synchronizer.

27. The system of claim 1, further comprising at least one digital-to-analog channel

28. A method for determining the neurological state of a subject, comprising the steps of:
providing an integrated system including an electrode array comprising at least one electrode, at least one analog amplifier channel, an analog-to-digital converter, a digital signal processor, and an output device; and
wherein the at least one analog amplifier channel, the analog-to-digital converter, and the digital signal processor are configured to reside in a single integrated physical circuit;
attaching the said at least one electrode to the subject; and
detecting signals relating to the subject's brain electrical activity,

29. The method of claim 28, wherein the at least one electrode detects analog brain electrical signals and transmits the signal to the digital processor via the at least one analog channel and the analog-to-digital converter.

30. The method of claim 28, further comprising the step of checking an electrode impedance.

31. The method of claim 28, further comprising the step of varying a sampling rate to avoid aliasing.

32. The method of claim 28, further comprising the step of administering stimuli to the brain using a stimulus generator.

33. The method of claim 32, further comprising the step of detecting evoked potentials generated in response to said stimuli.

34. The method of claim 28, further comprising the step of processing data relating to a subject's brain electrical activity

35. The method of claim 34, wherein said step of processing data utilizes harmonic signal analysis algorithm.

36. The method of claim 28, further comprising the step of transmitting a form of the subject's brain electrical activity to said output device.

37. The method of claim 36, wherein the step of transmitting the subject's brain electrical activity comprises transmission of unprocessed data.

38. The method of claim 36, wherein the step of transmitting the subject's brain electrical activity comprises transmission of processed data indicating the subject's neurophysiological condition.

39. A system for determining the neurological state of a subject comprising:
a single integrated physical circuit comprising at least one analog amplifier channel, an analog-to-digital converter, and a digital signal processor configured to perform a signal processing algorithm;
an electrode array comprising at least one electrode;
a stimulus generator configured to administer at least one of auditory, visual, and electrical stimulation.

40. The system of claim 39, further comprising an analog multiplexer.

41. The system of claim 39, wherein the said at least one electrode detects analog brain electrical signals and transmits the signals to said digital processor via said at least one analog amplifier channel, and said analog-to-digital converter.

42. The system of claim 41, wherein said digital signal processor processes said signals using harmonic analysis.

43. The system of claim 42, wherein said digital signal processor is further configured to perform fractal analysis and multiscale harmonic analysis.

44. The system of claim 39, wherein said digital signal processor outputs processed data related to a subject's neurological state.
